(19) United States
(12) Patent Application Publication
(10) Pub. No.: US 2021/0358218 A1
(43) Pub. Date: Nov. 18, 2021

(54) 360 VR VOLUMETRIC MEDIA EDITOR

(71) Applicant: Surgical Theater, Inc., Los Angeles, CA (US)

(72) Inventors: MORDECHAI AVISAR, HIGHLAND HEIGHTS, OH (US); Alon Yakob Geri, Orange Village, OH (US)

(21) Appl. No.: 17/278,302

(22) PCT Filed: Sep. 23, 2019
(86) PCT No.: PCT/US2019/052454

§ 371 Date: Mar. 20, 2021

Related U.S. Application Data
(60) Provisional application No. 62/735,616, filed on Sep. 24, 2018.

(51) Int. Cl. G06T 19/00; A61B 34/10; G06T 17/00; G06F 3/01; G06T 5/00; G16H 30/40; G16H 50/50

(52) U.S. Cl. CPC G06T 19/003; A61B 34/10; G06T 17/00; G06F 3/011; G06T 2219/004; G16H 30/40; G16H 50/50; A61B 2034/105; G06T 5/002

(57) ABSTRACT

A method includes obtaining medical images of the internal anatomy of a particular patient; preparing a three dimensional virtual model of the patient; generating a virtual reality environment using said virtual model of the patient to provide a realistic three dimensional images of actual tissues of the patient; providing an interface to receive user input defining a path through the internal anatomy of the patient within the virtual reality environment to capture various perspectives of the realistic three dimensional images of the internal anatomy of actual tissues of the patient; and generating a patient video capturing the defined path through the internal anatomy of the patient within the virtual reality environment, said patient video showing views of various perspectives of the realistic three dimensional images of the internal anatomy of actual tissues of the patient, said patient video being configured to play on a general purpose computing device.

### CROSS-REFERENCE TO RELATED APPLICATION

This application is a U.S. national stage application of PCT application serial number PCT/US2019/052454 filed on Sep. 23, 2019, which claims priority from U.S. provisional patent application Ser. No. 62/735,616 filed on Sep. 24, 2018 which is incorporated by reference herein in its entirety.

### FIELD OF DISCLOSURE

The present disclosure relates to the field of surgical procedures and more specifically to the field of surgical procedure preparation and education.

### BACKGROUND

When facing a complex surgical procedure, a patient may often experience fear and anxiety in the days and weeks leading up to the surgery. This may be the result of the patient not clearly understanding the procedure and therefore not knowing what to expect. Engaging the patient prior to the surgical procedure and educating the patient may help alleviate this fear and anxiety. Clearer communication between the treating physician and the patient pertaining the pathological situation of the patient and the proposed solution is important in overcoming uncertainties the patient may feel and in establishing trust between the patient on one hand, and the physician and the healthcare provider on the other. This is also important due to the competitive environment in which healthcare providers operate today, and the many options patients face in selecting physicians and providers. In addition, by engaging the patient and educating the patient about the procedure, the patient may be more likely to take appropriate care and steps to ensure a proper recovery without complications and without need for returning to the hospital for follow up care. Existing techniques for engaging and educating a patient, however, such as showing the patient an image of the anatomy or a 3D model, may not be effective, particularly when the surgery involves a part of the anatomy that is abstract or difficult to understand in the context of a standalone image or even a 3D model.

### SUMMARY

In one example, a method of preparing for a medical procedure includes the step of obtaining medical images of the internal anatomy of a particular patient. The method further includes preparing a three dimensional virtual model of the patient associated with the internal anatomy of the patient utilizing said medical images, The method further includes generating, using a computer device, a virtual reality environment using said virtual model of the patient to provide a realistic three dimensional images of actual tissues of the patient. The method further includes providing an interface on an input device of the computer device to receive user input defining a path through the internal anatomy of the patient within the virtual reality environment to capture various perspectives of the realistic three dimensional images of the internal anatomy of actual tissues of the patient. The method further includes generating a patient video capturing the defined path through the internal anatomy of the patient within the virtual reality environment, said patient video showing views of various perspectives of the realistic three dimensional images of the internal anatomy of actual tissues of the patient, said patient video being configured to play on a general purpose computing device. The method further includes transmitting said patient video to the general purpose computing device to play on said general purpose computing device.

In another example, a method of preparing for a medical procedure includes the step of obtaining medical images of the internal anatomy of a particular patient. The method further includes preparing a three dimensional virtual model of the patient associated with the internal anatomy of the patient utilizing said medical images. The method further includes generating, using a computer device, a virtual reality environment using said virtual model of the patient to provide realistic three dimensional images of actual tissues of the patient. The method further includes providing an interface on an input device of the computer device to receive user input defining a path through the internal anatomy of the patient within the virtual reality environment to capture various perspectives of the realistic three dimensional images of the internal anatomy of actual tissues of the patient. The method further includes generating a patient video capturing the defined path through the internal anatomy of the patient within the virtual reality environment, said patient video showing views of various perspectives of the realistic three dimensional images of the internal anatomy of actual tissues of the patient, said patient video being configured to play on a general purpose computing device. The method further includes transmitting said patient video to the general purpose computing device to play on said general purpose computing device for viewing by the patient. The method further includes the patient viewing said video on the general purpose computing device to prepare for the medical procedure.

In another example, a method of preparing for a medical procedure includes the step of obtaining medical images of the internal anatomy of a particular patient. The method further includes preparing a three dimensional virtual model of the patient associated with the internal anatomy of the patient utilizing said medical images. The method further includes generating, using a computer device, a virtual reality environment using said virtual model of the patient to provide realistic three dimensional images of actual tissues of the patient. The method further includes providing an interface on an input device of the computer device to receiving user input, including defining a path through the internal anatomy of the patient within the virtual reality environment to provide realistic three dimensional images of the internal anatomy of actual tissues of the patient, and accepting inputs from said input device to mark various locations along said path with a marker, wherein each of said markers can be associated with a particular perspective view of the realistic three dimensional images of the internal anatomy of actual tissues of the patient. The method further includes generating a patient video capturing the defined path through the internal anatomy of the patient within the virtual reality environment, said patient video showing views of various perspectives of the realistic three dimensional images of the internal anatomy of actual tissues of the patient. The video is generated using a smoothing operation to show a view that gradually transitions a change in perspective while the video traverses from the particular perspective view of one marker to the particular perspective view of an adjacent marker. The patient video is configured to play on a general purpose computing device. The method further includes transmitting said patient video to the general purpose computing device to play on said general purpose computing device for viewing by the patient. The method further includes the patient viewing said video on the general purpose computing device to prepare for the medical procedure.

### BRIEF DESCRIPTION OF THE DRAWINGS

In the accompanying drawings, structures are illustrated that, together with the detailed description provided below, describe exemplary embodiments of the claimed invention. Like elements are identified with the same reference numerals. It should be understood that elements shown as a single component may be replaced with multiple components, and elements shown as multiple components may be replaced with a single component. The drawings are not to scale and the proportion of certain elements may be exaggerated for the purpose of illustration.

FIG. 1 illustrates an example system for generating a custom 360 VR video fly-through of a virtual reality environment;

FIG. 2 is a block diagram of an example media editor computer of FIG. 1;

FIG. 3 illustrates an example graphical user interface provided by the example media editor computer of FIG. 1;

FIG. 4 illustrates an example user interface for enabling a physician to virtually enter a scene and to identify a path using a HMD;

FIG. 5 illustrates a perspective of view of a physician, depicted as an avator, as the physician virtually moves through a portion of a patient's body;

FIG. 6 illustrates an example user interface menu which may be activated for an icon while creating or editing a path

FIG. 7 is a flow chart of an example method for generating a custom 360 VR video fly-through of a virtual reality environment; and

FIG. 8 is a block diagram of an example computer for implementing an example media editor computer of FIG. 1;

### DETAILED DESCRIPTION

The following acronyms and definitions will aid in understanding the detailed description:

VR—Virtual Reality—A 3Dimensional computer generated environment which can be explored and interacted with by a person in varying degrees.

HMD—Head Mounted Display refers to a headset which can be used in VR environments. It may be wired or wireless. It may also include one or more add-ons such as headphones, microphone, HD camera, infrared camera, hand trackers, positional trackers etc.

SNAP Model—A SNAP case refers to a 3D texture or 3D objects created using one or more scans of a patient (CT, MR, fMR, DTI, etc.) in DICOM file format. It also includes different presets of segmentation for filtering specific ranges and coloring others in the 3D texture. It may also include 3D objects placed in the scene including 3D shapes to mark specific points or anatomy of interest, 3D Labels, 3D Measurement markers, 3D Arrows for guidance, and 3D surgical tools. Surgical tools and devices have been modeled for education and patient specific rehearsal, particularly for appropriately sizing aneurysm clips.

MD6DM—Multi Dimension full spherical virtual reality, 6 Degrees of Freedom Model. It provides a graphical simulation environment which enables the physician to experience, plan, perform, and navigate the intervention in full spherical virtual reality environment.

Fly-Through—Also referred to as a tour, it describes a perspective view of a virtual reality environment while moving through the virtual reality environment along a defined path.

A surgery rehearsal and preparation tool previously described in U.S. Pat. No. 8,311,791, incorporated in this application by reference, developed to convert static CT and MRI medical images into dynamic and interactive multi-dimensional full spherical virtual reality, six (6) degrees of freedom models (“MD6DM”) that can be used by physicians to simulate medical procedures in real time. The MD6DM provides a graphical simulation environment which enables the physician to experience, plan, perform, and navigate the intervention in full spherical virtual reality environment. In particular, the MD6DM gives the surgeon the capability to navigate using a unique multidimensional model, built from traditional 2 dimensional patient medical scans, that gives spherical virtual reality 6 degrees of freedom (i.e. linear; x, y, z, and angular, yaw, pitch, roll) in the entire volumetric spherical virtual reality model.

The MD6DM is built from the patient's own data set of medical images including CT, MRI, DTI etc., and is patient specific. A representative brain model, such as Atlas data, can be integrated to create a partially patient specific model if the surgeon so desires. The model gives a 360° spherical view from any point on the MD6DM. Using the MD6DM, the viewer is positioned virtually inside the anatomy and can look and observe both anatomical and pathological structures as if he were standing inside the patient's body. The viewer can look up, down, over the shoulders etc., and will see native structures in relation to each other, exactly as they are found in the patient. Spatial relationships between internal structures are preserved, and can be appreciated using the MD6DM.

The algorithm of the MD6DM takes the medical image information and builds it into a spherical model, a complete continuous real time model that can be viewed from any angle while “flying” inside the anatomical structure. In particular, after the CT, MRI, etc. takes a real organism and deconstructs it into hundreds of thin slices built from thousands of points, the MD6DM reverts it to a 3D model by representing a 360° view of each of those points from both the inside and outside.

A media editor described herein leverages a MD6DM model and enables a user to generate and share a custom 360 VR video “fly-through” of a portion of an anatomy according to a desired preselected path. For example, a physician may use the media editor to generate a custom “tour” that will lead a patient along a predefined path inside a portion of the inside of a body. The physician may present the video to the patient in an office setting or even outside of an office setting without relying on expensive surgery rehearsal and preparation tools. The physician may share the video with the patient, for example, in order to engage and educate the patient in preparation for a surgical procedure. The video may also be shared with other physicians, for example, for education and collaboration purposes. It should be appreciated that, although the examples described herein make specific reference to generating 360 VR videos of anatomy portions for the purpose of educating and collaborating between patients and medical professionals, 360 VR videos of other environments in various applications may similarly be generated and shared.

It should be appreciated that although specific references may be made to a physician, the media editor described herein may be used by any suitable user to generate and share a custom 360 VR video “fly-through” of a portion of an anatomy.

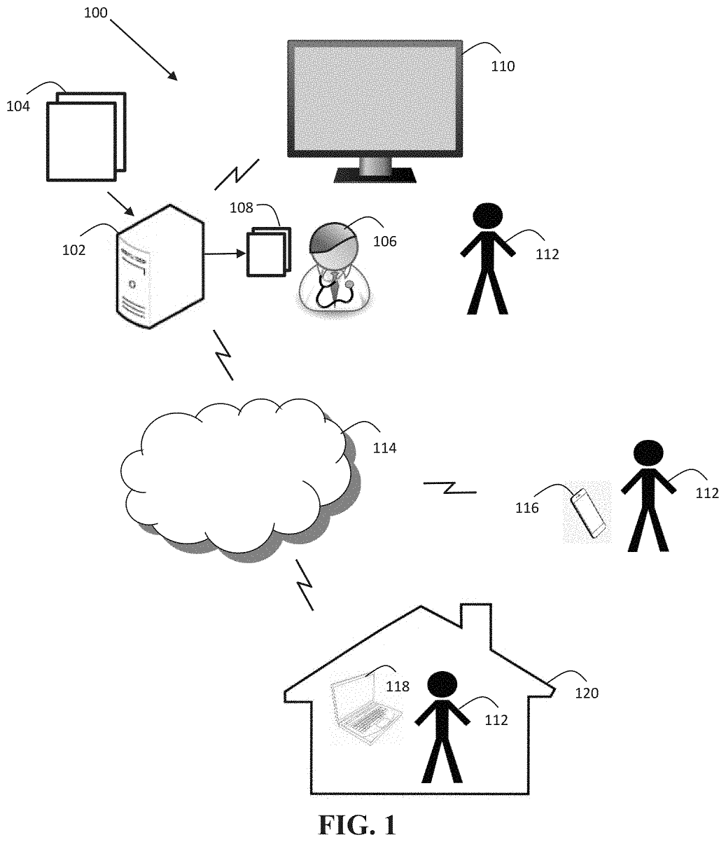

FIG. 1 illustrates an example system 100 for generating and sharing a custom 360 VR video “fly-through.” The system 100 includes a media editor computer 102 configured to receive inputs 104 such as MD6DM models or other suitable models or images corresponding to a virtual reality environment. The media editor computer 102 is further configured to enable a physician 106, or other suitable user, to interact with the inputs 104 via a user interface (not shown) and to generate a custom 360 VR video (“video”) 108 output including a fly-through of the virtual reality environment.

In one example, the media editor computer 102 is further configured to communicate the video 108 to a display 110, thus enabling the physician 106 to engage and interact with a patient 112, or any other suitable second user, as the video 108 is displayed on the display 110. In one example, the media editor computer 102 is further configured to enable the physician 106 to share the video 108 with the patient 112 remotely via a network 114. For example, the media editor computer 102 may enable the patient 112 to watch the video via mobile smartphone 116 or via a personal computer 118 in the patient's home 120.

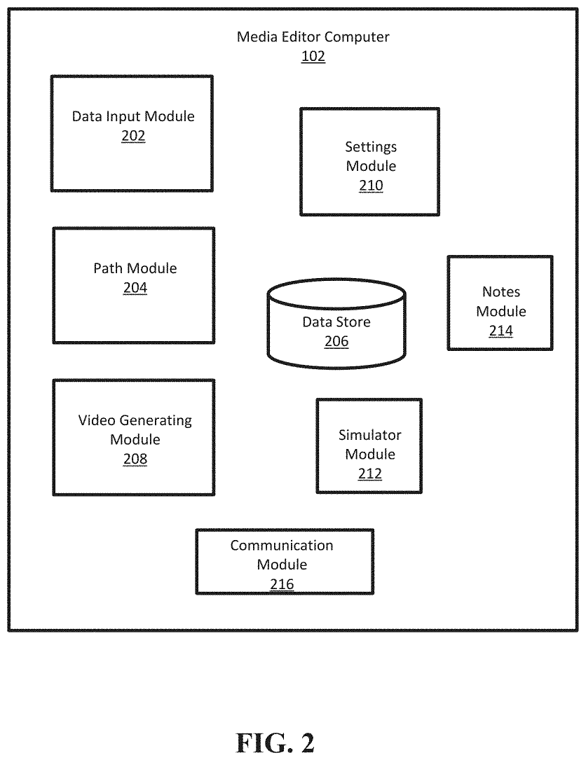

FIG. 2 illustrates the example media editor computer 102 of FIG. 1 in more detail. The media editor computer 102 includes a data input module 202 configured to communicate with data sources (not shown) and to receive the inputs 104 of FIG. 1 including a model representative of a virtual reality environment. In one example, the data input module 202 is configured to receive MD6DM models as input. In another example, the data input module 202 is configured to receive Mill scans, images from a video camera, of any suitable type of image data. The model representative of the virtual reality environment, will serve as a foundation based upon which the media editor computer 102 is configured to generate the video 108.

The media editor computer 102 further includes a path module 204 configured to load the model received by data input module 202 into a user interface and to enable the physician 106 to create a path for a fly through based on the inputs 104. A fly-through, also referred to as a tour, describes a perspective view of a virtual reality environment while moving through the virtual reality environment along the defined path.

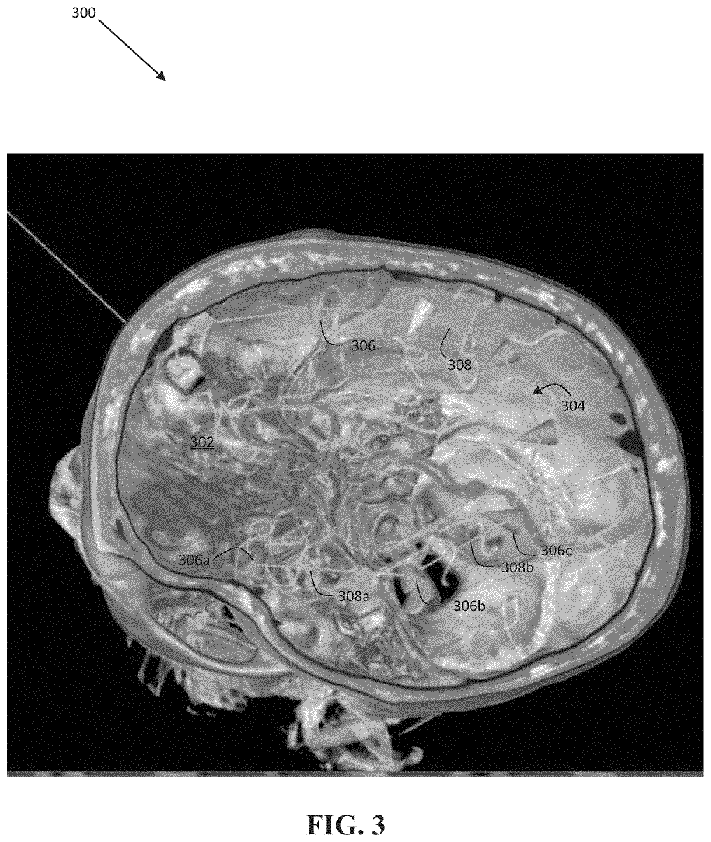

FIG. 3 illustrates an example media editor user interface 300 provided by the path module 204. The path module 204 is configured to display, via the media editor user interface 300, an image 302 representative of the virtual reality environment. It should be appreciated that, although the image 302 illustrated is representative of a brain, image 320 may include any suitable image representative of any suitable virtual reality environment such as a heart, a lung, and so on. It should be further appreciated that the image 302 may be a 2-dimensional image or the image 302 may be a 3D virtual reality environment.

The path module 204 is further configured to enable, via the media editor user interface 300, the physician 106 to identify a path 304 for the fly-through. In particular, the path module 204 is configured to enable, via the media editor user interface 300, the physician 106 to position a number of icons 306 on the image 302 to define the path 304. Specifically, the path module 204 is configured to receive input representative of a first icon 306a and a second icon 306b and to identify a first sub-path 308a between the first icon 306a and second icon 306b. The path module 204 is further configured to receive input representative of a third icon 306c and to identify a second sub-path 308b between the second icon 306b and third icon 306c. It should be appreciated that the path module 204 is configured to receive any suitable number of icons 306 and to generate a corresponding number of sub-paths 308, even though seven icons 306 and six sub-paths 308 are illustrated. The path module 204 is further configured to combine the first sub-path 308a, the second sub-path 308b, and any additional suitable sub-paths 308, to form the path 304.

In one example, the path module 204 is configured to receive, via the media editor user interface 300, the icons 306 via a drag-and-drop mechanism. For example, the media editor user interface 300 may enable the physician to select an icon 306 from a menu (not shown) and drag the icon 306 onto the image 302. It should be appreciated that other suitable use interface mechanisms may be used for placing icons 306 on the image 302.

In one example, the physician 106 may be provided with a HMD (not shown) for interacting with the user interface 300. For example, the path module 204 may enable the physician 106 to virtually enter a scene or virtual environment presented by the media editor user interface 300 using the HDM and to identify a path 304 by placing icons 306 along the path 304 as the physician 106 virtually moves through the anatomy. Such an example provides an immersive experience which may enable a physician 106 to more accurately define the path 304 since the physician 106 may have a point of view orientation such that may not otherwise be available when defining the path 304 via a 2-dimensional interface.

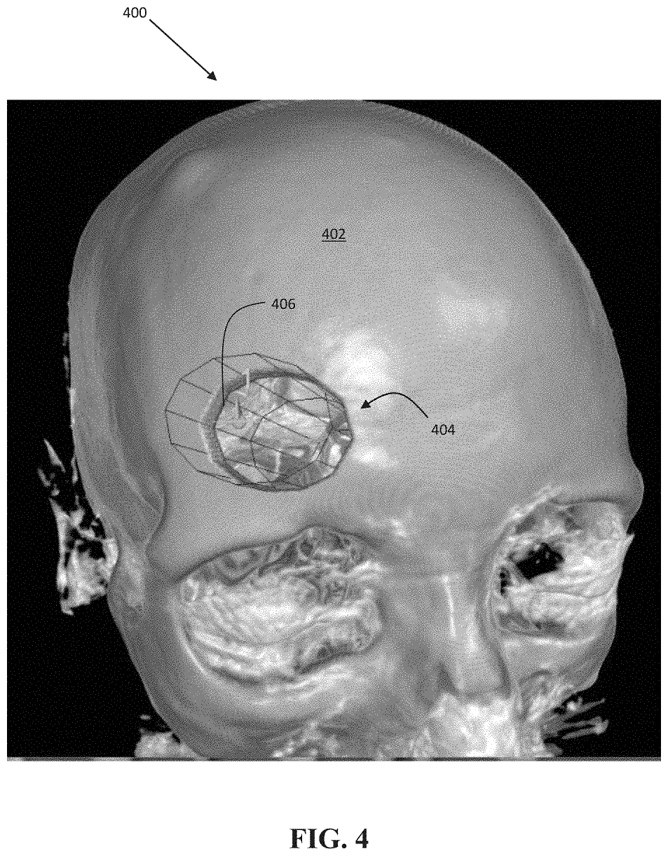

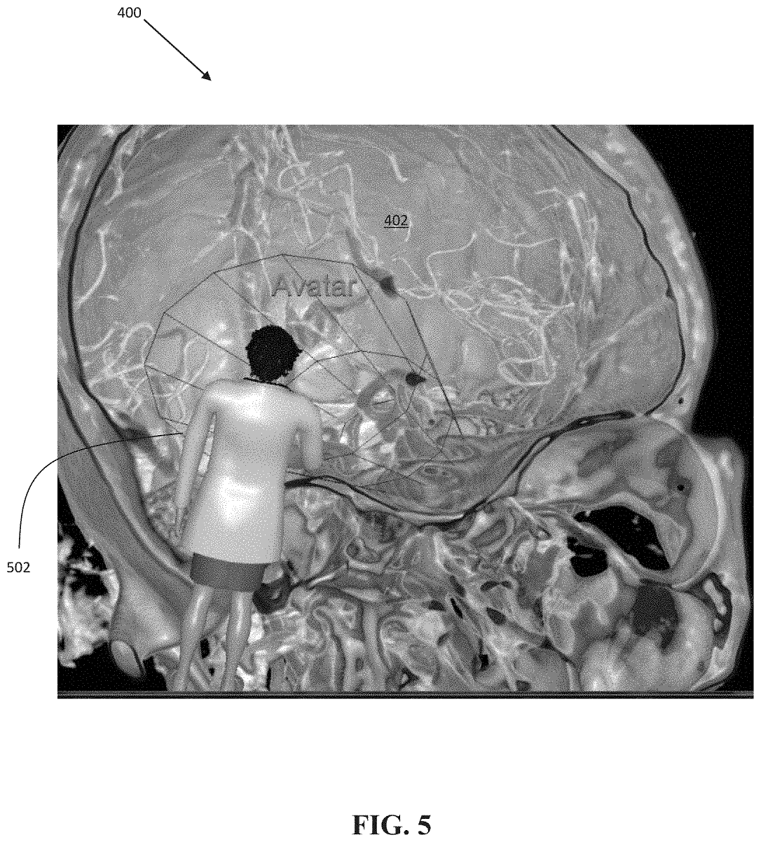

FIG. 4 illustrates an example user interface 400 for enabling a physician to virtually enter a scene and to identify a path using a HMD. For example, using the HMD, the physician may enter scene consisting of a skull 402 via a virtual opening 404 and place a first icon 406. The physician may then proceed to “fly” or virtually move through the skull 402 using the HMD to place additional icons in order to create path as previously described, while navigating through the skull 402 from a perspective of being physically inside the skull 402. In one example, as illustrated in FIG. 5, the perspective of view of a physician as the physician virtually moves through the skull 402 may be depicted by an avatar 502. The avatar 502 represents the virtual positon of the physician within the skull 402 as well as the physician's direction and angle of view. It should be appreciated that the avatar 502 may not be visible on the user interface 400 to the physician as the physician interacts with user interface 400 via the HMD. Rather, the avatar 502 may be displayed on a display device, other than the HMD. Thus, a second physician may follow along and potentially assist as the first physician navigates virtually through the skull 402.

Referring back to FIG. 2, the media editor computer 102 further includes a data store 206 configured to store data associated with the created path 304. In particular, the data store 206 is configured to store information about icons 306 and sub-paths 308 as the information is being received and generated by the path module 204. Thus, in one example, the media editor computer 102 enables a physician 106 to save progress of prior to completion of the video 108 and resume creation of the video 108 at a later point in time. In one example, the path module 204 is further configured to enable the physician to edit or delete information about the path stored in the data store 206.

The media editor computer 102 further includes a settings module 210 configured to enable the physician 106 to customize the fly-through for the entire path 304. For example, the settings module 210 may receive path settings via a user interface which may be initiated by a right click, a menu selection, and so on.

In one example, path settings received may include the speed at which the fly-through should occur in the video 108. In one example, the path settings received may further include an indication of whether the video 108 should be generated in an interactive 360-degree mode or in a passive two-dimensional mode. For example, in passive mode, the perspective of the virtual reality environment is fixed as the patient 112 is being guided along the path 304 of the virtual environment in a two-dimensional video. In one example, although the perspective is fixed in passive mode, the video may be generated as a three-dimensional stereoscopic video. In an interactive mode however, the patient 112 is able to choose the perspective of view as the patient 112 is being guided along the path 304 of the virtual environment in a 360-degree video. In other words, although the patient 112 is still directed along the defined path 304, the patient 112 may look wherever the patient 112 desires as the 36-degree video is being played for the patient 112.

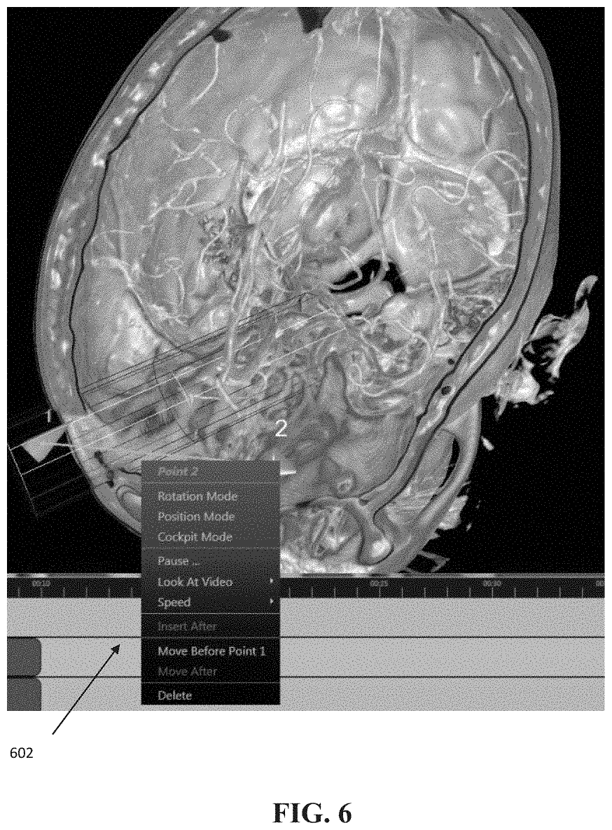

The settings module 210 is further configured to enable the physician 106 to customize the fly-through at each icon 306 individually through various icon settings. For example, the physician 106 may right click on an individual icon 306 in order to define one or more icon settings for the specific icon 306. FIG. 6 illustrates an example user interface menu 602 which may be activated for an icon while creating or editing a path. In one example, icon settings may include a speed setting. Although a path speed may be defined in the received path settings, a physician may choose to specify a certain portion of a video following a select icon to play at alternate speed and thus specify accordingly in an icon setting.

In one example, icon settings may include an orientation setting. For example, the settings module 210 may be configured to enable a physician to define the direction of the perspective view when positioned at a particular icon 306 along the path 304. Thus, the orientation may change as a patient 112 is flown along the path 304 between different icons 306. Enabling the orientation to change along the path 304 at the different icons 306 provides for the ability to direct focus as appropriate. In one example, icon settings may include an angle of view setting as well.

In one example, icon settings may include a layers setting. More specifically, a virtual reality environment may include multiple layers of view within the environment. For example, a virtual reality environment representative of a brain anatomy may include a bones layer, a blood vessels layer, and so on. The layers setting enables the physician 106 to turn off or turn on individual layers at each icon 306, thus enabling the physician 106 to direct what the patient 122 is able to view at each icon 306. In other words, it may be desirable to view all layers of a brain anatomy at the first icon 306a and to only view a subset of layers at the second icon 306b. In one example, it me be desirable to turn on or turn off a layer for the entire path 304. Accordingly, the path settings may also include a layers setting.

The settings module 210 is further configured to store the path settings and the icon settings in the data store 206. In one example, the settings module 210 is configured to enable the physician 106 to edit or delete settings stored in the data store 206.

The media editor computer 102 further includes a video generating module 208 configured to generate the video 108 including a fly-through of the virtual reality environment represented by the input 104, along the defined path 304 and based on the settings received by the settings module 210. In particular, the video generating module 208 generates the video 108 providing a perspective view of the virtual environment by simulating movement through the virtual reality environment along the defined path 304. In one example, the video generating module 208 is further configured to store the generated video 108 in the data store 206. It should be appreciated the video 108 may be created in any suitable video file format such as AVI, WMV, and so on.

In one example, the icon settings may include a fork setting. More specifically, the settings module 210 may enable the physician 106 to define a fork at an icon 306. That is, a patient 112 may be given an option to select from two or more paths to proceed with at a given icon 306. In such an example, multiple videos may be generated and stored in the data store 206. Accordingly, multiple videos may be linked together and presented to the patient sequentially based on selections made at respective icons 306.

In one example, the video generating module 208 is further configured to perform a smoothing operation when generating the video 108 along the path 304. More particularly, the video generating module 208 is configured to extrapolate information between the icons 306 in order to create for a more seamless and smooth movement between the icons 306. For example, the first icon 306a may be configured with a first orientation and the second icon 306b may be configured with a second orientation. Thus, when moving between the first icon 306a and the second icon 306b along the first sub-path 308a, the video generating module 208 is configured to gradually shift from the first orientation to the second orientation over the course of the first sub-path 308, instead of sharply transitioning between the first orientation and the second orientation at one icon 306. More particularly, the video generating module 208 is configured to determine the distance or time between the first icon 306a and the second icon 308b. The video generating module 208 is further configured to estimate a third orientation at some intermediate point in between the first icon 306a and the second icon 308b by extrapolating the first orientation and the second orientation over the determined distance or time. Thus, by transitioning from the first orientation to the third orientation before transitioning to the second orientation, the transition is perceived as smoother to the patient 112.

It should be appreciated that, although the smoothing operation has been described as extrapolating the first orientation at the first icon 306a and the second orientation at the second icon 306b over the determined distance or time to determine one additional third orientation at a single intermediate point in between the first icon 306a and the second icon 306b, any suitable number of intermediate points may be determined and used in between any of the icons 306 by the smoothing process. More particularly, using additional intermediate points may result in the transition being perceived as more smooth to the patient 112. It should be further appreciated that, although the smoothing process has been described with respect to orientation, smoothing may similarly be applied to other variables or settings. For example, the video generating module 208 may be further configured to perform the smoothing operation with respect to the position of the relative position of the icons 306. For example, the path 304 illustrated in FIG. 3 may be generally perceived as circular. However, the sub-paths 308 are linear. Thus, although the intention of the video may be to provide the patient 112 with a perception of a circular path 304, the patient 112 may perceive a linear, non-circular, motion along the individual sub-paths. Accordingly, the video generating module 208 may be further configured to extrapolate the relative positions of the icons 306 in order to determine the positioning along intermediate points in between the icons 306 in order to adjust the sub-paths 308 to become more rounded and provide the patient 112 with a smoother perceived transition.

The media editor computer 102 further includes a simulator module 212 configured to enable the physician 106 to switch to a preview mode or cockpit mode while editing the path 304 in order to preview the virtual reality view from the perspective of any of the icons 306. By being able to preview the virtual reality view in real time during the editing process, the physician 106 is able to fine tune the position and orientation of each icon 306 in order to achieve the precise desired view intended for the patient 112. In other words, the physician 106 is able to toggle between an edit mode and a preview or cockpit mode. In one example, the simulator module 212 is further configured to enable the physician 106 to preview the entire path 304 by flying between all of the icons 306. Thus, simulator module 212 enables the physician to preview the tour before the video is generated.

It should be appreciated that the physician 106 may preview the virtual reality view from the perspective of any of the icons 306, as described, either via the display 110 or an HMD (not shown). In one example, in addition to previewing the virtual reality view, the physician 106 may also edit the path 304 while previewing and flying through the path 304. For example, the physician 106 may add icons 306, remove icons 306, or reposition icons 306 in order to fine tune the path 304.

The media editor computer 102 further includes a notes module 214 configured to enable the physician 106 to add notes and other markups or additional data to the video at various points along the path 304. For example, the physician 106 may add a note describing a specific scene in the virtual reality environment associated with a specific icon 306 so that the patient 112 may review the note while viewing the video. The note may be written text, oral, or a graphic, for example. In one example, the notes module 214 is configured to store the notes in the data store 206. It should be appreciated that the notes module 214 enables the physician 106 to add notes along the path 304 either while creating the path using the path module 204 or any time thereafter before the video is generated by the video generating module 308.

In one example, the notes module 214 may enable the physician to associate questions or a test with the path 304 or with individual icons 306 in order to engage and educate patients 112 or students. In one example, the notes may be generated for marketing purposes. In other examples, the notes module 214 may enable the physician 106 to associate additional content such as videos or simulated surgical tools with the path 304 or with individual icons 306.

The media editor computer 102 further includes a communication module 216 configured to communicate the generate video 108 to the patient 112. In one example, the communication module 216 communicates the video 108 to the display 110 for immediate in person engagement and interaction between the physician 106 and the patient 112, at the physician's 106 office for example. In another example, the communication module 216 is configured to communicate the video 108 to the patient 112 remotely over the network 114. For example, the communication module 216 can be configured to transfer the video 108 over the network 114 to the patient 112 by email. In another example, the communication module 216 may be configured to communicate a link to the video 108 stored in the data store 206. The communication module 216 may communicate the link by email or by text message for example.

Once the video 108 is generated and shared, it can be used in a number of useful ways. For example, a patient may review the video at home with family in order to prepare for the surgery and explain to family what steps will be taken during the upcoming procedure. The patient may pause during the video 108 and point out certain areas of interest or answer specific questions. The patient may view the video on a smart phone, on a PC, or via a HMD, for example. The video 108 may also be used to educate other physicians or to collaborate with others. For example, a physician may use the video 108 to “walk” another physician through the anatomy and to describe specific features and make various points about a surgical procedure. In one example, the creator of the video 108 may add interactive features to the video 108 and provide the patient or other physician with an ability to customize the video fly through experience. For example, a patient may be provided with the option to select from different paths along the video or to turn on and off certain layers of the anatomy during the fly through. In one example, a patient may answer questions during the video fly through and submit answers to the physician in order to confirm understanding of the surgical procedure.

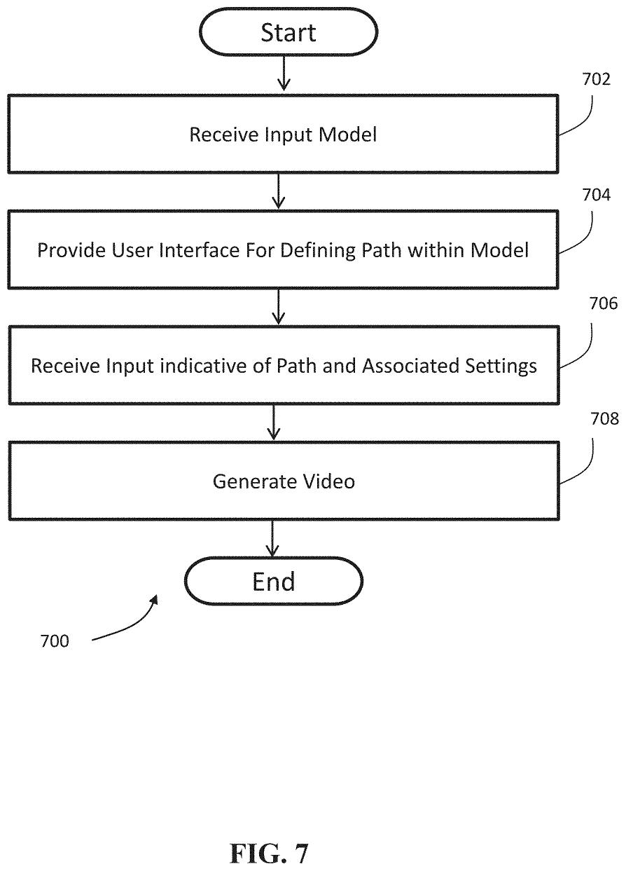

FIG. 7 illustrates an example method for generating a custom 360 VR video fly-through. At block 702, the media editor computer 102 receives input data including a model of a 3D virtual reality environment. At block 704, the media editor computer 102 provides a user interface for defining a path within the virtual reality environment. At block 706, the media editor computer 102 receives input indicative of the definition of the path and associated settings. Defining the path includes defining the steps or icons along the path while defining the settings includes defining the properties of the video at each step along the path. At block 708, the media editor computer 102 generates the video fly-through of the virtual reality environment and shares the video with a patient or other user.

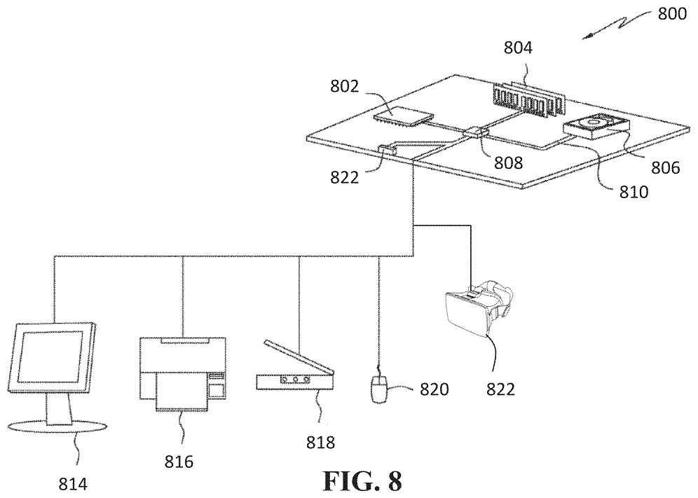

FIG. 8 is a schematic diagram of an example computer 800 for implementing the example media editor computer 102 of FIG. 1. The example computer 800 is intended to represent various forms of digital computers, including laptops, desktops, handheld computers, tablet computers, smartphones, servers, and other similar types of computing devices. Computer 800 includes a processor 802, memory 804, a storage device 806, and a communication port 808, operably connected by an interface 810 via a bus 812.

Processor 802 processes instructions, via memory 804, for execution within computer 800. In an example embodiment, multiple processors along with multiple memories may be used.

Memory 804 may be volatile memory or non-volatile memory. Memory 804 may be a computer-readable medium, such as a magnetic disk or optical disk. Storage device 806 may be a computer-readable medium, such as floppy disk devices, a hard disk device, optical disk device, a tape device, a flash memory, phase change memory, or other similar solid state memory device, or an array of devices, including devices in a storage area network of other configurations. A computer program product can be tangibly embodied in a computer readable medium such as memory 804 or storage device 806.

Computer 800 can be coupled to one or more input and output devices such as a display 814, a printer 816, a scanner 818, a mouse 820, and a HMD 822.

As will be appreciated by one of skill in the art, the example embodiments may be actualized as, or may generally utilize, a method, system, computer program product, or a combination of the foregoing. Accordingly, any of the embodiments may take the form of specialized software comprising executable instructions stored in a storage device for execution on computer hardware, where the software can be stored on a computer-usable storage medium having computer-usable program code embodied in the medium.

Databases may be implemented using commercially available computer applications, such as open source solutions such as MySQL, or closed solutions like Microsoft SQL that may operate on the disclosed servers or on additional computer servers. Databases may utilize relational or object oriented paradigms for storing data, models, and model parameters that are used for the example embodiments disclosed above. Such databases may be customized using known database programming techniques for specialized applicability as disclosed herein.

Any suitable computer usable (computer readable) medium may be utilized for storing the software comprising the executable instructions. The computer usable or computer readable medium may be, for example but not limited to, an electronic, magnetic, optical, electromagnetic, infrared, or semiconductor system, apparatus, device, or propagation medium. More specific examples (a non-exhaustive list) of the computer readable medium would include the following: an electrical connection having one or more wires; a tangible medium such as a portable computer diskette, a hard disk, a random access memory (RAM), a read-only memory (ROM), an erasable programmable read-only memory (EPROM or Flash memory), a compact disc read-only memory (CDROM), or other tangible optical or magnetic storage device; or transmission media such as those supporting the Internet or an intranet.

In the context of this document, a computer usable or computer readable medium may be any medium that can contain, store, communicate, propagate, or transport the program instructions for use by, or in connection with, the instruction execution system, platform, apparatus, or device, which can include any suitable computer (or computer system) including one or more programmable or dedicated processor/controller(s). The computer usable medium may include a propagated data signal with the computer-usable program code embodied therewith, either in baseband or as part of a carrier wave. The computer usable program code may be transmitted using any appropriate medium, including but not limited to the Internet, wireline, optical fiber cable, local communication busses, radio frequency (RF) or other means.

Computer program code having executable instructions for carrying out operations of the example embodiments may be written by conventional means using any computer language, including but not limited to, an interpreted or event driven language such as BASIC, Lisp, VBA, or VBScript, or a GUI embodiment such as visual basic, a compiled programming language such as FORTRAN, COBOL, or Pascal, an object oriented, scripted or unscripted programming language such as Java, JavaScript, Perl, Smalltalk, C++, Object Pascal, or the like, artificial intelligence languages such as Prolog, a real-time embedded language such as Ada, or even more direct or simplified programming using ladder logic, an Assembler language, or directly programming using an appropriate machine language.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995). Also, to the extent that the terms “in” or “into” are used in the specification or the claims, it is intended to additionally mean “on” or “onto.” Furthermore, to the extent the term “connect” is used in the specification or claims, it is intended to mean not only “directly connected to,” but also “indirectly connected to” such as connected through another component or components.

While the present application has been illustrated by the description of embodiments thereof, and while the embodiments have been described in considerable detail, it is not the intention of the applicants to restrict or in any way limit the scope of the appended claims to such detail. Additional advantages and modifications will readily appear to those skilled in the art. Therefore, the application, in its broader aspects, is not limited to the specific details, the representative apparatus and method, and illustrative examples shown and described. Accordingly, departures may be made from such details without departing from the spirit or scope of the applicant's general inventive concept.

What is claimed is:

1. A method of preparing for a medical procedure, the method comprising the steps of:
obtaining medical images of the internal anatomy of a particular patient;
preparing a three dimensional virtual model of the patient associated with the internal anatomy of the patient utilizing said medical images;
generating, using a computer device, a virtual reality environment using said virtual model of the patient to provide a realistic three dimensional images of actual tissues of the patient;
providing an interface on an input device of the computer device to receive user input defining a path through the internal anatomy of the patient within the virtual reality environment to capture various perspectives of the realistic three dimensional images of the internal anatomy of actual tissues of the patient;
generating a patient video capturing the defined path through the internal anatomy of the patient within the virtual reality environment, said patient video showing views of various perspectives of the realistic three dimensional images of the internal anatomy of actual tissues of the patient, said patient video being configured to play on a general purpose computing device; and
transmitting said patient video to the general purpose computing device to play on said general purpose computing device.

2. The method of claim 1, wherein said step of defining a path through the internal anatomy of the patient within the virtual reality environment includes the step of accepting inputs from said input device to mark various locations along said path with a marker.

3. The method of claim 2, wherein each marker is associated with a particular perspective view of the realistic three dimensional images of the internal anatomy of actual tissues of the patient.

4. The method of claim 3, wherein said video is generated using a smoothing operation to show a view that gradually transitions a change in perspective while the video traverses from the particular perspective view of one marker to the particular perspective view of an adjacent marker.

5. The method of claim 3, wherein said particular perspective view includes an angle of view and orientation within the virtual model.

6. The method of claim 2, wherein said step of defining a path through the internal anatomy of the patient within the virtual reality environment also includes the step of accepting inputs from said input device to associate each marker with one or more particular anatomy layers of the virtual model such that views of said layers in said patient video can be turned on and off along said path.

7. The method of claim 2, wherein said step of defining a path through the internal anatomy of the patient within the virtual reality environment also includes the step of accepting inputs from said input device to associate one or more of said markers with a speed of travel along a portion of said path in said patient video.

8. The method of claim 2, wherein said interface of said user input device is configured to provide the user with a drag-and-drop interface to place said markers along said path.

9. The method of claim 2, further comprising the step of providing a head-mounted display for use by the user to view said path to place said markers along said path.

10. The method of claim 2, wherein a marker is associated with a fork in the path splitting the path into two different paths providing a choice to viewer of said patient video to select one of the two different paths.

11. The method of claim 2, wherein a marker is associated with one or more notes or added data provided by the user using said interface.

12. The method of claim 11, wherein said notes include a question or a quiz.

13. The method of claim 2, wherein a marker is associated with a selectable control to provide an active video allowing a viewer to interact with a portion video or to provide a passive video that does not allow the viewer to interact with the portion of the video.

14. The method of claim 1, wherein a link is transmitted to said general computing device to download said patient video to said general computing device to play said video.

15. The method of claim 1, wherein said general purpose computing device is a smart phone.

16. The method of claim 1, wherein said step of defining a path through the internal anatomy of the patient within the virtual reality environment includes the step of accepting inputs from said input device to mark various locations along said path with a marker, wherein each marker is associated with a particular perspective view of the realistic three dimensional images of the internal anatomy of actual tissues of the patient.

17. A method of preparing for a medical procedure, the method comprising the steps of:
obtaining medical images of the internal anatomy of a particular patient;
preparing a three dimensional virtual model of the patient associated with the internal anatomy of the patient utilizing said medical images;
generating, using a computer device, a virtual reality environment using said virtual model of the patient to provide realistic three dimensional images of actual tissues of the patient;
providing an interface on an input device of the computer device to receive user input defining a path through the internal anatomy of the patient within the virtual reality environment to capture various perspectives of the realistic three dimensional images of the internal anatomy of actual tissues of the patient;
generating a patient video capturing the defined path through the internal anatomy of the patient within the virtual reality environment, said patient video showing views of various perspectives of the realistic three dimensional images of the internal anatomy of actual tissues of the patient, said patient video being configured to play on a general purpose computing device;
transmitting said patient video to the general purpose computing device to play on said general purpose computing device for viewing by the patient; and
the patient viewing said video on the general purpose computing device to prepare for the medical procedure.

18. A method of preparing for a medical procedure, the method comprising the steps of:
obtaining medical images of the internal anatomy of a particular patient;
preparing a three dimensional virtual model of the patient associated with the internal anatomy of the patient utilizing said medical images;
generating, using a computer device, a virtual reality environment using said virtual model of the patient to provide realistic three dimensional images of actual tissues of the patient;
providing an interface on an input device of the computer device to receiving user input including the steps of:
defining a path through the internal anatomy of the patient within the virtual reality environment to provide realistic three dimensional images of the internal anatomy of actual tissues of the patient, and
accepting inputs from said input device to mark various locations along said path with a marker, wherein each of said markers can be associated with a particular perspective view of the realistic three dimensional images of the internal anatomy of actual tissues of the patient;
generating a patient video capturing the defined path through the internal anatomy of the patient within the virtual reality environment, said patient video showing views of various perspectives of the realistic three dimensional images of the internal anatomy of actual tissues of the patient, wherein
said video is generated using a smoothing operation to show a view that gradually transitions a change in perspective while the video traverses from the particular perspective view of one marker to the particular perspective view of an adjacent marker, and wherein
said patient video is configured to play on a general purpose computing device;
transmitting said patient video to the general purpose computing device to play on said general purpose computing device for viewing by the patient; and
the patient viewing said video on the general purpose computing device to prepare for the medical procedure.

19. The method of claim 18, wherein the user interface is configured to accept associating one or more of said markers with one or more of: a speed of travel along a portion of said path in said patient video, one or more layers of anatomy to show along a portion of said path in said patient video, or a fork in the path splitting the path into two different paths providing a choice to patient to select one of the two different paths while viewing said video.

20. The method of claim 18, the user interface is configured to accept associating one or more of said markers with a selectable control to provide an active video allowing the patient to interact with a portion video or to provide a passive video that does not allow the patient to interact with the portion of the video.
